(19) United States
(12) Patent Application Publication
(10) Pub. No.: US 2011/0009833 A1
(43) Pub. Date: Jan. 13, 2011

(54) IMPLANTABLE SITE WITH SCREEN ENGAGING IN THE SEPTUM

(75) Inventor: Pascal Paganon, Serezin du Rhone (FR)

Correspondence Address: OCCHIUTI ROHLICEK & TSAO, LLP, 10 FAWCETT STREET, CAMBRIDGE, MA, 02138, US

(73) Assignee: COMPAGNIE EUROPEENNE D'ETUDE ET DE RECHERCHE DE DISPOSITIFS POUR L'IMPLANTATION PAR LAPAROSCOPIE, Vienne (FR)

(21) Appl. No.: 12/920,378

(22) PCT Filed: Mar. 5, 2009
(86) PCT No.: PCT/FR2009/050368

§ 371 Date: Aug. 31, 2010

(30) Foreign Application Priority Data
Mar. 6, 2008 (FR) FR-08 01237

(51) Int. Cl. A61M 39/04; B23P 11/00

(52) U.S. Cl. 604/288.02; 29/428

(57) ABSTRACT

The invention relates to an implantable medical device designed for the injection and/or removal of a fluid substance into and/or from a human or animal body, said device comprising a housing (2) provided with a chamber (3) that extends between a first end and a second end and that is delimited by a side wall (6) which joins said first end to said second end, said side wall (6) having at least a first puncture zone (10) and a second puncture zone (11), said device comprising a non-pierceable screen (13), said device being characterized in that the side wall (6) is formed in one piece, and in that a portion of the non-pierceable screen (13) is inserted into a blind seat (14) formed within the thickness of said side wall (6). Implantable medical devices.

### TECHNICAL DOMAIN

The present invention pertains to the general technical domain of devices designed to be introduced beneath the skin of a human patient or animal patient so that these devices can be subsequently pierced with a hollow needle, through the patient's skin, for the purpose of introducing substances into and/or removing substances from said patient's body while at the same time restricting injuries related to repeated injections.

The present invention pertains more particularly to an implantable medical device designed for the injection and/or removal of a fluid substance into and/or from a human or animal organism, said device comprising a box provided with a chamber designed to receive said fluid substance, said chamber extending between a first end and a second end and being delimited by a side wall which joins said first end to said second end, said side wall having at least one first puncture zone and one second puncture zone facing one another and designed so as to be capable of being pierced by a needle, said device comprising a non-piercable screen positioned in the chamber in order to prevent a needle that penetrates said chamber through the first puncture zone from leaving the chamber by again piercing the side wall through the second puncture zone.

The present invention also pertains to a method for manufacturing an implantable medical device designed to inject a fluid substance into a human or animal organism and/or remove a fluid substance therefrom.

### PRIOR ART

There are known ways of implanting a medical device, known as an implantable site, beneath a patient's skin designed to form an access point placed at a distance, enabling the transfer of substances into or from the blood circulation system or the tissues of an organ or again an inflatable implant such as a balloon catheter or a constriction ring.

Generally, an implantable site of this kind takes the form of a box in which there is prepared a chamber delimited by a wall, said wall being at least partly conceived to be pierced by a hollow needle designed to transfer a fluid into said chamber by injection or by extraction.

To this end, the wall usually has an puncture zone formed by a self-plugging membrane or “septum” made out of an elastomer material in such a way that the imperviousness of the site is preserved equally well when the needle crosses the wall and when it is withdrawn, and the hole formed by the needle gets closed automatically, when this needle is withdrawn, through an elastic self-healing phenomenon.

Generally, the chamber communicates with a flexible catheter fixed to the box in such a way that the injection zone can be moved away to an anatomical region that is more easily accessible than the destination zone or the source zone of the transferred fluid.

Now it has been noted that, once implanted, the prior art sites were liable to overturn under the effect of stress or shock.

In order to spare the patient a fresh surgical operation to reposition the site, an implantable site has been conceived with a chamber delimited by a perforation-resistant cylindrical wall plugged at each of its ends by a distinct circular septum.

Thus, a site of this kind takes the form of a disk with two opposite puncture zones, one on its upper face and the other on its lower face, in such a way that when one of said puncture zones rests on the subcutaneous tissues, the other puncture zone substantially faces the patient's skin.

Advantageously, the chamber can therefore remain accessible if the site should be overturned.

However, this arrangement of the puncture zones exposes the site to being pierced from one side to the other, resulting in a malfunctioning of the operation of extraction or injection, or even injury to the subcutaneous tissues.

This is why, in order to prevent the needle that penetrates the chamber from again crossing the wall of the chamber substantially opposite its entry point, it has been proposed to place a perforation-resistant screen in the chamber to obstruct the section of said chamber so as to block the needle.

Although such a site has generally been satisfactory in terms of comfort and injection safety, both for the patient and for the practitioner, it may all the same have certain non-negligible drawbacks.

Indeed, in order to ensure the mechanical strength of the site as well as its lasting imperviousness, the self-sealing membranes forming puncture zones are generally relatively thick.

Moreover, said self-sealing membranes frequently have a small length so that they can be kept appropriately under compression in a housing made in the box.

Consequently, such sites, on the whole, have a useful surface and accessibility that are relatively limited when compared with their overall dimensions.

Furthermore, the non-piercable screens of the prior art may have moving or deformable parts which, because of the repeated stresses to which they are subjected by the needles, can sometimes get blocked or break causing irreparable malfunctioning of the site.

### SUMMARY OF THE INVENTION

The objects assigned to the present invention are therefore aimed at remedying the above-mentioned drawbacks and proposing a novel implantable medical device designed for the injection and/or removal of fluids into and/or from a human or animal organism that has excellent accessibility while at the same time being particularly sure and reliable in its implementation.

Another object assigned to the invention is aimed at proposing a novel implantable medical device that possesses high robustness and great longevity.

Another object assigned to the invention is aimed at proposing a novel implantable medical device having a particularly simple, compact and light structure.

Another object assigned to the invention is aimed at proposing a novel implantable medical device that is comfortable and non-traumatic for the patient.

Another object assigned to the invention is aimed at proposing a method for making an implantable medical device designed for the injection and/or removal of a fluid substance into and/or from a human or animal organism that gives said device high accessibility and great reliability of functioning.

Finally, another object assigned to the invention is aimed at proposing a new method for manufacturing an implantable medical device that is particularly simple and costs little to implement.

The objects assigned to the present invention are obtained by means of an implantable medical device designed for the injection and/or removal of a fluid substance into and/or from a human or animal organism, said device comprising a box provided with a chamber designed to receive said fluid substance, said chamber extending lengthwise between a first end and a second end along a longitudinal axis (X-X′) said chamber being delimited by a side wall which joins said first end to said second end, said side wall having at least one first puncture zone and one second puncture zone situated so as to be facing each other and designed to be capable of being pierced by a needle, said device comprising a non-piercable screen positioned in the chamber in order to prevent a needle that penetrates said chamber through the first puncture zone from coming out of the chamber in again piercing the side wall through the second puncture zone, said device being characterized in that the side wall is formed in one piece lengthwise along the longitudinal axis (X-X′) and in that a portion of the non-piercable screen is inserted into a blind housing hollowed out in the thickness of said side wall.

The objects of the invention are also attained by means of a method for making an implantable device designed for the injection and/or removal of a fluid substance into and/or from a human or animal organism, said method comprising a step (a) for making a box provided with a chamber designed to receive said fluid substance during which a chamber extending is delimited lengthwise along a longitudinal axis (X-X′) between a first end and a second end by means of a side wall which presents at least one first puncture zone and one second puncture zone situated so as to be facing each other and designed to be capable of being pierced by a needle, said method comprising a step (b) for mounting a non-piercable screen during which a non-piercable screen is positioned in the chamber in order to prevent a needle that penetrates said chamber through the first puncture zone from coming out of the chamber in again piercing the side wall through the second puncture zone, said method being characterized in that the step (a) for making the box comprises a sub-step (a₁) for making the side wall during which the side wall is made in one piece lengthwise along the longitudinal axis (X-X′) and a blind housing is hollowed out in the thickness of said side wall and in that the step (b) for mounting the non-piercable screen comprises an insertion step (b₁) in which a portion of the non-piercable screen is engaged in said blind housing.

### BRIEF DESCRIPTION OF THE DRAWINGS

Other objects, features and advantages of the invention shall appear in greater detail from the following description as well as from the appended drawings, given purely on an illustrative and non-restrictive basis, of which:

FIG. 1 is an illustration, in an exploded perspective view, of an alternative embodiment of an implantable medical device according to the invention.

FIG. 2 is a view in perspective of a part of the implantable medical device shown in FIG. 1, when it is assembled.

FIG. 3 is a longitudinal cut-away view in perspective of the alternative embodiment of the implantable medical device corresponding to FIGS. 1 and 2 when said medical device is assembled.

### PREFERRED EMBODIMENT OF THE INVENTION

The present invention pertains to an implantable medical device 1 designed for the injection and/or removal of fluid substances into and/or from a human or animal organism.

A device 1 such as this, also called an “implantable site”, is designed to be implanted surgically in a patient's body, preferably beneath said patient's skin, in order to form a point of access for the introduction and/or extraction of fluid substances into and/or from said patient's body.

Said device 1 can be applied and adapted to different uses.

In particular, the device 1 of the invention can be designed for the injecting and/or extraction of fluid into and/or from an organ or the circulatory system, for example to enable the injection of medicinal substances. According to a particular variant of this application, said device 1 can be adapted to the formation of an artificial vein or artery which the practitioner can then pierce through the skin, as with a natural vein, in order to inject a therapeutic substance or remove blood.

The device 1 of the invention can also be adapted to feeding implanted containers associated for example with insulin or antalgic pumps.

The device 1 finally can be adapted to the injection and removal of a fluid into and from the inflatable compartment of a surgical implant, such as an artificial sphincter, a balloon catheter or again a gastric ring designed to constrict the stomach in order to combat obesity.

Here below, the device 1 shall be considered more particularly to be a hypodermic device, i.e. a device designed to be positioned just beneath the patient's skin, although said device can also be implanted in other parts of the patient's body and at a deeper level without departing from the scope of the invention.

According to the invention, the device 1 has a box 2 provided with a chamber 3 which itself is designed to receive the injected or removed fluid substance.

Said chamber 3 extends between a first end 4 and a second end 5 and is delimited by a side wall 6 which joins said first end 4 to said second end 5.

Advantageously, the side wall 6 has at least one first puncture zone 10 and one second puncture zone 11 which are situated so as to be facing each other, said first and second puncture zones 10, 11 being conceived so that they can be pierced by a needle 12.

Naturally, the side wall 6 is capable of providing imperviousness to the device 1 at said first and second puncture zones 10, 11, in doing so both during piercing by the needle 12 and consecutively at the withdrawal of said needle 12, the hole made by the piercing of the wall 6 with the needle 12 automatically closing again by elastic self-healing after this needle is withdrawn.

To this end, the side wall 6 will preferably be made out of an advantageously biocompatible elastomer material of the silicone type. The side wall 6 will therefore preferably form a septum.

Besides, this device 1 has a non-piercable screen 13 positioned in the chamber 3 and more particularly between the first and second puncture zones 10, 11, in order to prevent a needle 12 that penetrates said chamber through the first puncture zone 10 from going out of the chamber in again piercing the side wall 6 through the second puncture zone 11.

Indeed, the expression “situated so as to be facing each other” indicates the fact that the first puncture zone 10 and the second puncture zone 11 are, as understood in the invention, arranged in such a way that it is geometrically possible, through the chamber 3, to plot at least one direct trajectory between an entry point situated in the first puncture zone 10 and a substantially opposite exit point situated in the second puncture zone 11, i.e. that it would be geometrically possible that, when there is no non-piercable screen 13 according to the invention, the needle 12 penetrating the chamber 2 by the first puncture zone 10 will cross the device from one side to the other in coming out of the box 2 by the second puncture zone 11.

According to one major characteristic of the invention, the side wall 6 is formed out of a single piece and a portion of the non-piercable screen 13 is inserted into a blind housing 14 which is hollowed out in the thickness of said side wall 6.

In other words, the device 1 advantageously has a monoblock side wall 6 that is permeable to perforation and has at least one recess opening out into the chamber 3, said recess being positioned so as to locally thin out said side wall 6 which, besides, preferably has a substantially constant thickness, and so as to receive the non-piercable screen 13 in permitting this screen to penetrate the thickness of said side wall 6.

The term “formed by a single piece” or “monoblock” indicates the fact that the side wall 6 extends uninterruptedly from the first end 4 up to the second end 5.

The side wall 6 could advantageously form a monoblock and homogenous septum that is preferably unique.

Preferably, the ratio between the depth P of the housing 14 and the total thickness E of the wall ranges substantially from 60% to 90%.

Advantageously, the layout of the non-piercable screen 13 according to the invention prevents not only the piercing of the box 2 on either side through the chamber 3, whatever the incidence at which the needle 12 penetrates the chamber 3, but also the tangential piercing of said box 2 which can occur when the needle 12 engages the side wall 6 tangentially and moves forward in its thickness without penetrating the chamber 3 itself.

In other words, the layout of the device 1 and more particularly of the screen 13 relative to the lateral wall 6 also enables the screen to intercept a needle 12 and block its progress when said needle 12 travels for a very long time within said side wall 6 without in any way coming out of it to penetrate the interior of the chamber 3.

Advantageously, we thus prevent an accidental tangential piercing in which the needle 12 would remain blocked in the septum without enabling the injection or removal of fluid or in which this needle would cross said septum from one side to another and emerge into the tissues, injuring them. Thus, a use that is both sure and reliable is guaranteed for the device 1.

Moreover, the layout of the puncture zones 10, 11 advantageously provides access to the chamber 3 in a plurality of directions of approach, providing for high accessibility to the site.

Preferably, with the chamber 3 stretching substantially along the longitudinal axis (X-X′) that joins the first end 4 to the second end 5, as illustrated in the figures, the cumulated angular cover of the first and second puncture zones 10, 11 about the longitudinal axis (X-X′), considered in a projection plane normal to said longitudinal axis (X-X′), is greater than or equal to 90 degrees and preferably greater than or equal to 180 degrees, and, in a particularly preferable way, substantially equal to 360 degrees. The septum forming the side wall 6 can thus take the shape of a tubular sleeve surrounding the chamber 3.

Preferably, said longitudinal axis (X-X′) corresponds to the mean direction of extension of the box 2, the chamber 3, the side wall 6 and the non-piercable screen 13 between the first and second ends.

According to a preferred embodiment, the chamber 3 and the non-piercable screen 13, and more comprehensively the box 2, extend substantially in parallel to the longitudinal axis (X-X′).

Furthermore, said longitudinal axis (X-X′) is preferably rectilinear and advantageously corresponds to the generating axis of revolution of the device 1, so that the side wall 6 and more generally the box 2 substantially shows a geometry of revolution.

Naturally, the shape of the box 2 and of the side wall 6 is in no way restricted and can also take a pear-shaped, ellipsoidal, barrel-shaped or other geometry.

However, as illustrated in the figures, the pack 2 and the side wall 6 will preferably take the shape of a circular-based right cylinder with an axis (X-X′).

In this respect, it must be noted that the box 2 and/or the chamber 3 can advantageously have a ratio between their overall length, measured along the longitudinal extension axis (X-X′) and their overall cross dimension measured orthogonally to said longitudinal axis (X-X′) appreciably ranging from 1.2 to 1.5.

More particularly, the side wall 6 takes the shape of a right cylinder, where the ratio of its length L to its diameter D ranges appreciably from 1.2 to 1.5.

Thus, the device 1 compliant with the invention and more particularly the chamber 3 and the side wall 6 advantageously have an shape elongated along the longitudinal axis (X-X′), i.e. the sense of their greatest extent, hence their length, is substantially the same and substantially coincides with said longitudinal axis (X-X′).

The side wall 6 thus extends in a single piece lengthwise substantially along said longitudinal axis (X-X′) in a direction that is substantially normal to its thickness, said side wall 6 having a length L greater than or equal to and preferably strictly greater than its overall cross-wise dimension. Similarly, the chamber 3 and the device 1 are advantageously more extended along the longitudinal axis (X-X′) than they are cross-wise to this axis.

In this respect, it can be noted that those skilled in the art could easily adapt the length of the device to the application in view, and especially implement very long devices and chambers, even of small diameter, i.e. devices that are appreciably longer than they are wide, without this being detrimental to their imperviousness, accessibility or reliability of operation.

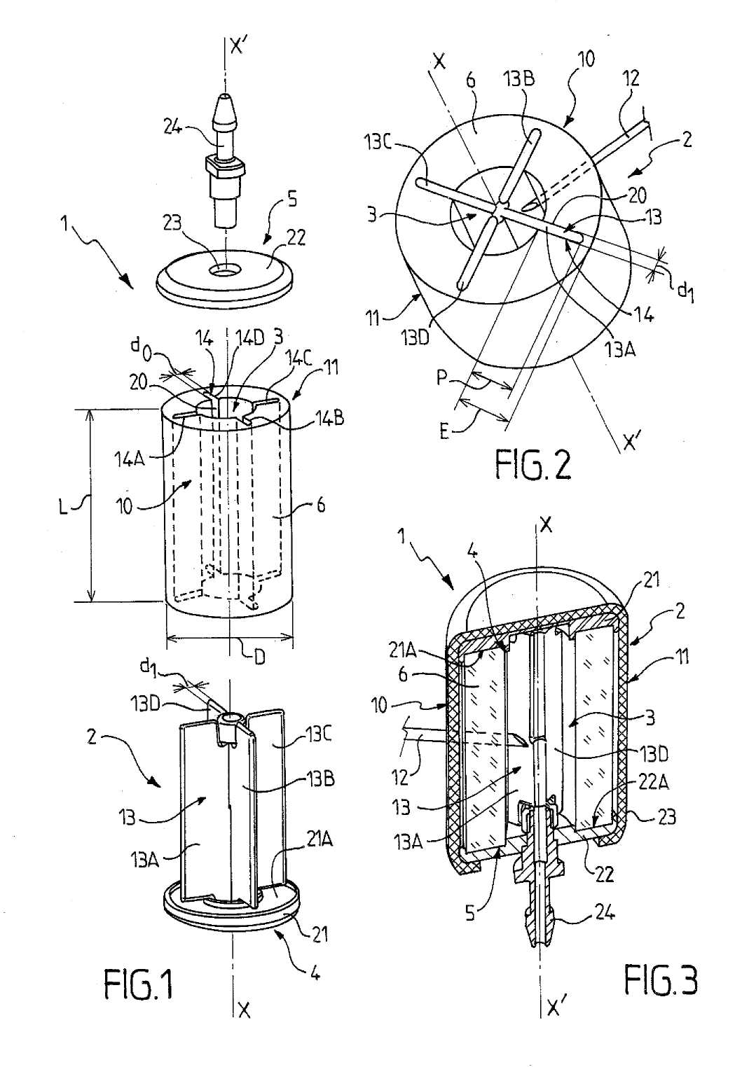

As shown in FIGS. 1 and 2, the housing 14 is preferably formed by a groove 20 substantially parallel to the longitudinal axis (X-X′).

Naturally, said groove 20 will take a shape substantially matching that of the non-piercable screen portion 13 housed therein.

In a particularly preferable way, as shown in FIG. 1, said groove will form a vertical trench hollowed out of the inner face of the side wall 6.

In addition, the groove 20 extends preferably over at least 50% of the length L of the side wall 6 measured between the first end 4 and the second end 5, preferably over at least 75% of this length L and even more preferably over the totality of said length L.

Thus, advantageously, the non-piercable screen 13 will cover the entire length of the side wall 6, thus not only improving the stability of the entire unit by maximising the supporting surface between the non-piercable screen 13 and the side wall 6 but also guaranteeing the reliability of operation of the site 1 by preventing an accidental tangential piercing on the entire side wall 6.

In addition, opening the groove 20 on the edges of the side wall 6, i.e., especially on the borders of the septum which extend to the first and second ends 4, 5 substantially perpendicularly to the longitudinal axis (X-X′), advantageously simplifies the assembling through the nesting of the non-piercable screen 13 within the housing 14.

Furthermore, said non-piercable screen 13 is preferably mounted so as to be tight in the housing 14, i.e. force-fitted in such a way that the material of the side wall 6 intimately matches the surface of the portion of the non-piercable screen 13 which is inserted into said housing 14.

Advantageously, this can be obtained by simple elastic tightening giving the groove 20 a form matching that of the screen 13 whose width at rest d0 is smaller than or equal to the corresponding thickness di of the non-piercable screen 13. Thus, the side wall 6 can envelop said non-piercable screen 13 without any play.

Furthermore, the non-piercable screen 13 preferably forms a rigid frame that supports the side wall 6.

To this end, said non-piercable screen could advantageously be made out of a rigid or semi-rigid material, and especially out of a plastic material such as poly(ether-ether-ketone) (PEEK), polycarbonate (PC), polysulphone (PSU) or poly-oxy-methylene (POM).

Thus, the non-piercable screen 13 could form a sort of pillar against which the side wall 6 gets supported, giving the device 1 high robustness and enabling it to preserve substantially the functional volume of the chamber 3, thus guaranteeing the efficiency of the transfer of fluid and efficient circulation of the fluid thus removed or injected.

Preferably, as illustrated in the figures, the non-piercable screen 13 will comprise a plurality of blades, 13A, 13B, 13C, 13D that meet in the chamber 3 to form a star structure.

In particular, said star could comprise three or preferably four arms and could especially be made with two panels each having two blades fixedly joined to each other, said panels being slit in the middle so that they fit into each other so as to as to form a cross-piece as illustrated in FIG. 1.

Thus, the device 1 has a structure that is particularly simple, compact and light.

Preferably, the blades 13A, 13B, 13C, 13D will take the form of flat, rigid or semi-rigid and preferably rectangular plates.

Preferably, said blades will be positioned so as to be substantially parallel to the longitudinal axis (X-X′), and oriented in directions that are radial relative to this axis, so as to meet one another other substantially at the centre of the chamber at a common captive edge.

In this constructional arrangement, the respective unattached edges of the blades 13A, 13B, 13C, 13D, i.e. the edges furthest away from the longitudinal axis (X-X′) forming the points of the star, can advantageously form the portions of the non-piercable screen that penetrate the thickness of the side wall 6, substantially on the rim of the box 2.

Thus, the respective unattached edges of several blades 13A, 13B, 13C, 13D constituting the star and preferably the respective unattached edges of all of said blades 13A, 13B, 13C, 13D are each inserted into a corresponding housing 14A,14B, 14C, 14D which is prepared in the thickness of the side wall 6.

In other words, the device 1 will preferably have as many housings 14A,14B, 14C, 14D as there are branches in the star.

Advantageously, a constructional arrangement of this kind enables a reciprocal holding of the non-piercable screen 13 and the side wall 6 and, therefore, a mutual immobilizing of these elements, thus improving the mechanical strength of the implantable site 1.

Furthermore, in a particularly preferable way, the housings 14A, 14B, 14C, 14D will all be substantially identical, as also the blades 13A,13B, 13C, 13D, in such a way that the implantable site 1 substantially shows a rotational invariance and especially fourth-order rotational invariance when the screen has four arms.

Advantageously, the manufacture of the elements can be thus standardized and the production costs can be reduced accordingly.

Furthermore, the first end 4 is preferably formed by a first perforation-resistant flange 21.

Similarly, the second end 5 is preferably formed by a second perforation-resistant flange 22.

Advantageously, the first and the second flanges 21, 22 could take the form of rigid or semi-rigid disks made out of a perforation-resistant material and laid out substantially perpendicularly to the longitudinal axis (X-X′).

Preferably, as shown in FIG. 3, the first and/or second flange 21, 22 could advantageously comprise a dip 21A, 22A forming a mounting feature that holds the side wall 6.

Moreover, the second flange 22 will preferably be pierced with a hole 23 receiving a tip 24 to which a catheter (not shown) can be attached so as to make the chamber 3 communicate with said catheter and thus enable the implantable site 1 to be moved away relative to the target puncture injection zone which is the destination zone or source zone of the transferred fluid.

Preferably, as shown in FIGS. 1 and 3, the first flange 21, the side wall 6, the second flange 22 and the tip 24 are aligned in this order, so as to be substantially coaxial along the longitudinal axis (X-X′).

In a preferred alternative embodiment, the site 1 has an external sheath 23 preferably with rounded contours, made out of an elastomer material piercable by a needle and is designed to complete the finishing of the site.

Furthermore, in one embodiment, not shown, the non-piercable screen 13 could form one piece with the first flange 21 or with the second flange 22.

A monoblock sub-unit of this kind, joining the non-piercable screen 13 and the first flange 21, can advantageously be obtained by moulding, especially by injection moulding both in the case of a plastic material and that of a metallic material (using the MIM process). This also greatly simplifies the operation for assembling the implantable side 1, since it is enough then to insert the side wall 6 by engaging the blades 13A, 13B, 13C, 13D, thus pre-positioned by construction, into their respective housings 14A, 14B, 14C, 14D until the edge of said side wall 6 is brought against the flange 21.

In this respect, it is noteworthy that the invention also pertains to a method for manufacturing an implantable device 1 designed to for the injection and/or the extraction of a fluid substance into and/or from a human or animal organism.

The method of the invention comprises a step (a) for making a box 2 provided with a chamber 3 that is designed to receive said fluid substance, this being a step during which a chamber 3 extending between a first end 4 and a second end 5 is delimited by means of a side wall 6 having at least one first puncture zone and one second puncture zone situated so as to be facing each other and conceived so as to be piercable by a needle 12.

Preferably, to this end, a first flange and a second flange 21, 22, for example circular shaped, are made out of a perforation-resistant material, as is also a cylindrical side wall 6 made out of a material piercable by a needle, said flanges 21, 22 being then attached to the edges of this side wall 6.

Said method also comprises a step (b) for mounting a non-piercable screen during which a non-piercable screen 13 is placed in the chamber 3 so as to prevent a needle 12 that penetrates said chamber 3 through the first puncture zone 10 from coming out of the chamber in again piercing the side wall 6 through the second puncture zone 11.

According to a major characteristic of the invention, the step (a) for making the box comprises a sub-step (a₁) for making the side wall during which the side wall 6 is made as a single piece and a blind housing 14 is hollowed out in the thickness of said side wall 6.

According to another major characteristic of the invention, the step (b) for mounting the non-piercable screen comprises an insertion sub-step (b₁) during which a portion of the non-piercable screen 13 is engaged in said blind housing 14.

Preferably, with the non-piercable screen 13 comprising a plurality of blades 13A, 13B, 13C, 13D laid out in the form of a star so as to be substantially parallel to a longitudinal axis (XX′) as already described here above and the side wall comprising a plurality of blind housings 14A, 14B, 14C, 14D having shapes matching those of the unattached edges of said blades, and said housings 14A, 14B, 14C, 14D taking the form of grooves substantially parallel to said longitudinal axis (XX′), the insertion sub-step (b₁) is made by inserting the side wall 6 into the non-piercable screen 13 in the direction of the longitudinal axis (XX′) by simultaneously engaging the unattached edges of the blades 13A, 13B, 13C, 13D in their respective grooves.

This operation is advantageously facilitated when the non-piercable screen 13 forms one piece with the first flange 21 in such a way that said flange 21 can serve as a grasping means to hold the screen 13 in position, while the side wall 13 is made to gradually slide until its upper edge is made to abut the flange 21, and more particularly the bottom of the dip 21A.

The force-fitting of the screen into the side wall 6 advantageously provides an elastic pre-stressing of said side wall 6 in forcing the lips of the grooves 20 to move apart by wedge effect so that the contour of the housings 14A, 14B, 14C, 14D intimately, and without play, matches the corresponding surfaces of the non-piercable screen 13.

This link between the screen 13 and the side wall 6 can advantageously be reinforced by bonding.

It is noteworthy that, in one alternative mode of implementation of the method, the sub-step (a₁) for making the side wall 6 can advantageously be made by extrusion of an elastomer through a head with a profile similar to the profile of a notched belt, so as to make it possible to produce cylindrical pre-grooved side walls 6, of any unspecified length, without there being any need to attach material, for example by bonding. As a variant, this making sub-step (a₁) can be done by moulding.

Thus, the implantable site 1 of the invention is advantageously accessible at any point about its longitudinal extension axis (XX′) while ensuring the reliability and safety of the act of injection since it prevents the needle 12 from crossing the box 2 from one part to the other whatever the orientation of approach of the needle.

Furthermore, the site according to the invention advantageously has a simple structure made of elements that cost little to make and are easy to assemble, ensuring that it is compact, robust and light.

### POSSIBILITY OF INDUSTRIAL APPLICATION

The invention finds industrial application in the designing and making of implantable sites.

1. Implantable medical device designed for the injection and/or removal of a fluid substance into and/or from a human or animal organism, said device comprising a box provided with a chamber designed to receive said fluid substance, said chamber extending lengthwise between a first end and a second end along a longitudinal axis (XX′), said chamber being delimited by a side wall which joins said first end to said second end, said side wall having at least one first puncture zone and one second puncture zone situated so as to be facing each other and designed to be capable of being pierced by a needle, said device comprising a non-piercable screen positioned in the chamber in order to prevent a needle that penetrates said chamber through the first puncture zone from coming out of the chamber in again piercing the side wall through the second puncture zone, wherein the side wall is formed in one piece lengthwise along the longitudinal axis (XX′) and a portion of the non-piercable screen is inserted into a blind housing hollowed out in the thickness of said side wall.

2. Device according to claim 1 wherein, with the chamber and the non-piercable screen, extending substantially in parallel to the longitudinal axis (XX′) which joins the first end to the second end, the housing is formed by a groove substantially parallel to said longitudinal axis (XX′).

3. Device according to claim 2 wherein the groove extends preferably over at least 50% of the length (L) of the side wall measured between the first and second end, preferably over at least 75% of this length (L) and even more preferably over the totality of this length (L).

4. Device according to claim 1 wherein the non-piercable screen is mounted tightly into the housing.

5. Device according to claim 1, the ratio between the depth (P) of the housing and the thickness (E) of the side wall ranges substantially from 60% to 90%.

6. Device according to claim 1 the first end is formed by a first perforation-resistant flange and/or in that the second end is formed by a second perforation-resistant flange.

7. Device according to claim 6 wherein the non-piercable screen forms one piece with the first flange or with the second flange.

8. Device according to claim 1 wherein the non-piercable screen comprises a plurality of blades that meet in the chamber to form a star structure.

9. Device according to claim 8 wherein the respective unattached edges of several blades constituting the star and preferably the respective unattached edges of all of said blades are each inserted into a corresponding housing which is prepared in the thickness of the side wall.

10. Device according to claim 1 wherein the side wall is made of a biocompatible elastomer material of the silicone type.

11. Device according to claim 1 wherein the non-piercable screen preferably forms a rigid frame that supports the side wall.

12. Device according to claim 1 wherein, with the chamber stretching substantially along an longitudinal axis (XX′), the cumulated angular cover of the first and second puncture zones about the longitudinal axis (XX′), considered in a projection plane normal to said longitudinal axis (XX′) is greater than or equal to 90 degrees, and preferably greater than or equal to 180 degrees, and, in a particularly preferable way, appreciably equal to 360 degrees.

13. Device according to claim 1 wherein the side wall presents substantially a geometry of revolution.

14. Device according to claim 1 wherein ratio between the overall length of the side wall, measured between the first end and the second end, and the overall width of said side wall substantially ranges from 1.2 to 1.5.

15. Method for manufacturing an implantable device designed for the injection and/or removal of a fluid substance into and/or from a human or animal organism, said method comprising a step (a) for making a box provided with a chamber designed to receive said fluid substance in which a chamber is delimited extending lengthwise along a longitudinal axis (X-X′) between a first end and a second end by means of a side wall which presents at least one first puncture zone and one second puncture zone situated so as to be facing each other and designed to be capable of being pierced by a needle, said method comprising a step (b) for mounting a non-piercable screen during which a non-piercable screen is positioned in the chamber in order to prevent a needle that penetrates said chamber through the first puncture zone from coming out of the chamber in again piercing the side wall through the second puncture zone, said method being characterized in that the step (a) for making the box comprises a sub-step (a₁) for making the side wall during which the side wall is made in one piece lengthwise along the longitudinal axis (XX′) and a blind housing is hollowed out in the thickness of said side wall and in that the step (b) for mounting the non-piercable screen comprises an insertion step (b₁) in which a portion of the non-piercable screen is engaged in said blind housing.

16. Method according to claim 15 wherein, with the non-piercable screen comprising a plurality of blades laid out in the form of a star so as to be substantially parallel to a longitudinal axis (XX′) and the side wall comprising a plurality of blind housings having shapes matching those of the unattached edges of said blades, said housings taking the form of grooves substantially parallel to said longitudinal axis (XX′), the insertion sub-step (b₁) is made by inserting the side wall into the non-piercable screen in the direction of the longitudinal axis (XX′) by simultaneously engaging the unattached edges of the blades in their respective grooves.
